(19) United States
(12) Patent Application Publication
(10) Pub. No.: US 2025/0122106 A1
(43) Pub. Date: Apr. 17, 2025

(54) CHEMICAL REDUCTION PROCESS OF PFAS CONTAINED IN WATER

(71) Applicant: PERMA-FIX ENVIRONMENTAL SERVICES, INC., Atlanta, GA (US)

(72) Inventors: Jose Gabriel GARCIA, Atlanta, GA (US); Louis F. CENTOFANTI, Atlanta, GA (US); Tommy W. YARBROUGH, Atlanta, GA (US)

(21) Appl. No.: 18/888,593

(22) Filed: Sep. 18, 2024

Related U.S. Application Data
(60) Provisional application No. 63/655,844, filed on Jun. 4, 2024.
(60) Provisional application No. 63/555,113, filed on Feb. 19, 2024.
(60) Provisional application No. 63/602,736, filed on Nov. 27, 2023.
(60) Provisional application No. 63/544,544, filed on Oct. 17, 2023.

(51) Int. Cl. C02F 1/58; C02F 1/02; C02F 1/52; C02F 101/36; C02F 103/06

(52) U.S. Cl. CPC C02F 1/583; C02F 1/02; C02F 1/5245; C02F 2101/36; C02F 2103/06; C02F 2305/023

(57) ABSTRACT

A system and method for degrading and incinerating a fluorocarbon or fluorinated material, such as per- and/or polyfluoroalkyl substances (PFAS), contained in water, such as ground water or leachate, with reduced emissions of gaseous PFC is provided. The method includes mixing the water, a hydroxide base, and optionally a solvent in a batch reactor to form a suspension. The PFAS and solvent can both be provided by the water, such as ground water or leachate. The reaction mixture is heated to a temperature ranging from about 25° C. to about 400° C. for at least about 0.5 hours to about 240 hours to defluorinate the corresponding PFAS fluorocarbons contained in the leachate water and produce a defluorinated waste product. The method can convert organic fluorine present in the PFAS within the water to inorganic fluoride. Thus, the defluorinated waste product can be incinerated with reduced emissions of harmful gaseous PFCs.

### CROSS-REFERENCE TO RELATED APPLICATIONS

This U.S. non-provisional patent application claims the benefit of and priority to U.S. provisional patent application Ser. No. 63/544,544, filed Oct. 17, 2023; U.S. provisional patent application Ser. No. 63/602,736, filed Nov. 27, 2023; U.S. provisional patent application Ser. No. 63/555,113, filed Feb. 9, 2024; and U.S. provisional patent application Ser. No. 63/655,844, filed Jun. 4, 2024, the entire disclosures of which are hereby incorporated by reference in their entirety.

### BACKGROUND OF THE INVENTION

### 1. Field of the Invention

The current invention relates generally to a system and method for degrading and disposing of fluorocarbon or fluorinated materials, for example per- and polyfluoroalkyl substances (PFAS) contained in contaminated water (“leachate water”).

### 2. Background Art

There are a variety of examples where per- and poly-fluoroalkyl substances (PFAS) has have contaminated water, including ground water, leachate out of landfills, municipal water, industrial water, hazardous water, and water from a variety of other sources. One significant concern relates to landfill leachate.

Widespread disposal of landfill leachate to municipal sewer infrastructure in the United States calls for an improved understanding of the relative organic-chemical contributions to the wastewater treatment plant (WWTP) waste stream and associated surface-water discharge to receptors in the environment. (1).

In the United States, disposal of municipal solid and liquid waste from residential, commercial, and industrial sources in landfills continues to increase in response to population growth and expanded manufacturing and availability of consumer products. (2)

Leachate is produced at landfills from the percolation of precipitation through solid waste and from liquid waste migrating down gradient. Complex mixtures of contaminants of concern to human and ecosystem health, including per- and poly-fluoroalkyl substances (PFAS), (3-10), are increasingly detected in leachate due to the expanded availability of personal-care products and packaging of single-use items and containers. (11, 12) PFAS are used in a wide range of consumer products such as electronics, water-repellent textiles, food packaging materials, carpets, and upholsteries that are commonly discarded into landfills. (10, 13) PFAS, which are largely resistant to biotic transformations due to their extremely strong C—F bonds, have been shown to cause disruption to key cellular functions. PFAS can also cause negative biological effects in animals and humans exposed to PFAS at high levels. (14-18) Exposure of PFAS even at low concentrations is an environmental concern, as they exhibit long biological half-lives and bioaccumulation potential. The annual leachate load of PFAS from U.S. landfills to municipal wastewater treatment plant (WWTP) influent was estimated to be between 563 and 638 kg in 2013. (7)

Adverse environmental effects from exposure to complex contaminant mixtures, such as PFAS at low ng L⁻¹ concentrations are currently unknown or inadequately characterized. (19, 20). In the United States, landfill leachate is primarily discharged to sewer infrastructure for co-treatment in WWTPs, (6) which are well-documented sources of organic contaminants in the environment. (21-31) Landfill leachate disposal rates (<0.1 to 2.0 million L d⁻¹) (6) are considerably lower (approximately 1% by volume) than WWTP influent rates (8 to 1300 million L d⁻¹). (25-29, 31. 32) However, leachate has been reported to contain substantially elevated concentrations of organic chemicals, such as PFAS. Several examples include perfluorooctanoic acid (PFOA); perfluorohexanoic acid (PFHxA); perfluoroheptanoic acid (PFHpA); perfluorooctanesulfonate (PFOS); perfluorohexanesulfonate (PFHxS); and methyl perfluoropentane sulfonamido acetic acid (MeFPeSAA).

Several existing leachate treatment systems are k-new known. Conventional wastewater treatment systems, such as activated carbon (AC) adsorption, flocculation, biological treatment, and membrane separation, have been employed at landfills to treat leachate before discharging the leachate to the municipal wastewater treatment plants or natural waterbodies. Although they may not have been designed for PFAS treatment, some existing leachate treatment facilities may be able to reduce the PFAS concentrations in leachate to various degrees. (33)

A variety of ground water sources have been contaminated by AFFF firefighting foams and factory discharges which contain PFAS. These materials can also be treated. Presently, other processes are being used and tested, including activated charcoal and critical water oxidation (CWO). The inventive process disclosed herein could be used with CWO to improve the efficiency of the CWO unit.

Fluorocarbons and fluorinated substances, such as per- and polyfluoroalkyl substances (PFAS), are anthropogenic synthetic materials used for over 90 years (34) and found in water at harmful concentrations to humans (35). PFAS have been designated as emerging contaminants of concern since 2000 (36). They can bioaccumulate in humans (37, 38, 39), have been linked to certain cancers, and have a wide range of deleterious effects including hormone and immune system interferences, ulcerative colitis, and endocrine disruption (40, 41, 42), to name a few.

PFAS compose a diverse class of chemicals that, due to their low surface tension and wetting properties, are found in a wide range of products and processes, including fluoropolymers, liquid repellants for paper, packaging, textile, leather, and carpet goods, industrial surfactants, additives, coatings, and firefighting foams. (43) Fluoropolymers, for example polytetrafluoroethylene (PTFE), are believed to be the most commonly found PFAS for computer applications and surface coatings.

The United States Environmental Protection Agency (USEPA) CompTox database has identified over 9000 highly fluorinated substances with Chemical Abstracts Service numbers available in the global market, the majority being fluorinated polymers and fluorinated surfactants (44).

The most recent guideline effective on Jun. 25, 2024. has advisory limits of 4 parts per trillion (ppt) of PFOA, (45, 46) and PFOS, the USEPA added five more PFAS compounds for site cleanups (perfluoronanoic acid (PFNA), PFHxS, perfluorononanoate, perfluorooctanoate, and perfluorohexanesulfonate) based on risk-based values for regional screening levels (RSLs) (36).

Various techniques have been used to decompose or dispose of hazardous substances, such as PFAS by electrochemical oxidation, direct irradiation, plasma treatment, photocatalysis, sonolysis, supercritical water oxidation, reductive hydrodefluorination and thermal degradation/incineration (47).

In 2020, the U.S. EPA (48) published a technical brief on the incineration of PFAS with the main conclusion that the effectiveness of incineration in destroying PFAS and their fate in terms of potential mixed fluorinated organic byproduct formation is not clearly understood. A significant concern is that incomplete destruction of PFAS can result in the formation of PIC (products of incomplete combustion), e.g., smaller PFAS molecules, which could be a potential hazard. Only a few studies are available related to PFAS incineration in full-scale operating facilities (48, 50-52). According to Solo-Gabriele et al., increasing incinerator temperatures decreases the total treated PFAS concentrations. However, not all PFAS species decreased with increasing temperatures (50). There is an alarming report of higher concentrations of PFOA found in the air at the incinerator sites compared to upwind sites (51). Public concern is that the incineration may spread PFAS and not break them down. This publication claims that the preliminary data show that soil and surface water near a commercial facility in Cohoes, New York, that has burned firefighting foam containing PFAS, are contaminated with PFAS (53).

PFAS incineration can occur directly for PFAS-based materials, such as firefighting foams or indirectly via the incineration of waste containing PFAS, such as textiles, etc. (54) Recently the Defense Department issued a ban on incinerating PFAS-laden items, with particular emphasis on the aqueous film-forming foam often used in training and combat situations (55). In addition, under the 2022 National Defense Authorization Act (56), the military is now prohibited from incinerating PFAS-containing materials in accordance with the Clean Air Act (57). Most incineration studies monitored a limited number of compounds, leaving the question of “unmonitored” PFAS unanswered (58). Even though multiple studies were done on the thermal degradation of PFAS (59-63), only limited data (64-65) is available on directly detecting degradation products during field-scale incineration. The main obstacle is still the lack of both suitable emission sampling methods (including industrial field sampling) to capture PFAS compounds and analytical methods to identify/detect PFAS and their thermal decomposition byproducts. The question remains unanswered as to how significant is the portion of volatile species that escape the analysis.

Thermal degradation/incineration is the widely available approach for managing contaminated solids, liquids, or gases using already built incinerator facilities (66), and many incineration facilities are already knowingly or unknowingly treating PFAS (e.g., consumer products, activated carbon regeneration). As previously mentioned, incineration facilities have already been deployed and are well-established in the industry.

Therefore, the initial cost of implementing the new technology disclosed here will be significantly reduced compared to other PFAS-destructive technologies. Together, these advantages place them as a critical solution for managing PFAS-containing waste (67-68).

However, in general, waste byproducts in any incineration include bottom ash, which contains non-combusted products, and gas, containing tiny particles and volatile products (67). According to Wang et al. (67) regarding PFAS incineration, the resulting ash and gas are both problematic. Ash contains inorganic fluorine and remaining PFAS bound to inorganic compounds such as calcium. Ash is typically sent to a landfill or repurposed. Particulates in the gas can be captured with electrostatic precipitators. However, HF is anticipated to be the main product of PFAS thermal conversion during incineration and is a corrosive/acidic gas. Capturing or removing volatile fluoride-containing byproducts may also be problematic.

Any untreated PFAS or byproducts from incineration are released directly into the environment (34). Therefore, the potential risk of secondary air and soil pollution and the return of PFAS into the environment is very high. In addition, incomplete destruction during thermal treatment/incineration could generate an unknown array of byproducts, which might be environmentally problematic. Since current knowledge of the fate of PFAS is limited, there is concern that PFAS incineration can release toxic gases, for example tetrafluoromethane, hexafluoroethane, fluoro-dioxins, fluoro-benzofurans, and perfluorinated carboxylic acids (69-70).

Thermal treatment, such as incineration, is a popular and effective technique used to dispose of hazardous substances in land-scarce and resource-lacking locations because it can greatly reduce volume of the waste and can produce electricity. However, a significant concern associated with incineration of the waste is emission of harmful gases. Dioxin and furan are commonly studied gases because they are serious health hazards. There are few studies on the production of perfluorinated compound (PFC) gases from the thermal treatment process. However, it was reported that thermal decomposition of PFAS can form gas-phase PFCs, such as CF₄ and C₂F₆, which are harmful to the environment. Gaseous PFCs are potent greenhouse gases. The global warming potential of CF₄ is 6500 times that of CO₂, and the atmospheric lifetime of C₂F₆ is 50,000 years. Due to the long atmospheric lifetime of gaseous PFCs, gaseous PFC emissions can permanently alter the radiative budget of the atmosphere. Other methods for disposing PFAS are being investigated, but fluorinated halogenated hydrocarbons, such as those found in PFAS, are very difficult to destroy due to the strength of the carbon-fluorine bond. There remains a need for a more sustainable method for disposing of PFAS.

### SUMMARY

One aspect of the disclosure provides a method for disposing a perfluoroalkyl or polyfluoroalkyl substance (PFAS) contained in water, such as leachate. The method comprises the steps of placing the water containing the PFAS, a hydroxide base, and optionally a solvent system in a batch reactor to form a suspension, and heating the suspension in the batch reactor to produce a defluorinated waste product.

Another aspect of the disclosure provides a system comprising a batch reactor for disposing the water containing the PFAS. The method includes placing the water containing the PFAS, a hydroxide base, and optionally a solvent system in the batch reactor to form a suspension, and heating the suspension in the batch reactor to produce a defluorinated waste product.

### BRIEF DESCRIPTION OF THE DRAWINGS

Other advantages of the present invention will be readily appreciated, as the same becomes better understood by reference to the following detailed description when considered in connection with the accompanying drawing.

FIG. 1 illustrates an example batch system for defluorinating a fluorocarbon present in PFAS, specifically PFOA, to produce a defluorinated waste product according to the following equation: 15KOH+C₈F₁₅O₂H→15KF+formate+carbonate+oxalate+glycolate.

### DETAILED DESCRIPTION OF EXAMPLE EMBODIMENTS

The materials, compounds, compositions, and methods described herein may be understood more readily by reference to the following detailed description of specific aspects of the disclosed subject matter and the examples included therein.

Before the present materials, compounds, compositions, and methods are disclosed and described. It is to be understood that the aspects described below are not limited to specific methods of specific reagents, as such may, of course, vary. It is also to be understood that the terminology used herein is for the purpose of describing only and is not intended to be limiting.

Also, throughout this specification, various publications are referenced. The disclosures of these publications are hereby incorporated by reference into this application in order to more fully describe the state of the art to which the disclosed matter pertains.

In this specification and the claims that follow, reference will be made to several terms, which shall be defined to have the following meanings. Throughout the specification and claims the word “comprise” and other forms of the word, such as “comprising” and “comprises,” means including but not limited to, and is not intended to exclude, for example, other additives, solvents, bases, components, integers, or steps. As used herein, the singular forms “a,” “an,” and “the” include plural referents unless the context clearly dictates otherwise. Thus, for example, reference to “a composition” includes mixtures of two or more such compositions. “Optional” or “optionally” means that the subsequently described event or circumstance can or cannot occur, and that the description includes instances where the event or circumstance occurs and instances where it does not. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms. As used herein, the term “composition” is intended to encompass a product comprising the specified ingredients in the specified amounts, as well as any product which results, directly or indirectly, from combination of the specified ingredients in the specified amounts. References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture. A weight percent (wt. %) of a component, unless specifically stated to the contrary, is based on the total weight of the formulation or composition in which the component is included. As used herein, the term “substituted” is contemplated to include all permissible substituents of inorganic base compounds. In a broad aspect, the permissible substituents include all alkali and alkaline-earth metals in the periodic table. Illustrative substituents include, for example, those described below. The permissible substituents can be one or more and the same or different for appropriate inorganic base compounds.

Those people of ordinary skill in the art will appreciate that Compounds of Formula I are examples of inorganic base analogs. As used herein, “an analog of potassium hydroxide” or “analogs of potassium hydroxide” are not limited to those analog compounds represented by Formula I, and may include many additions or substitutions of elements, groups, or moieties to the chemical structure of potassium hydroxide.

M(OH)x (Formula I)
wherein x is the number of hydroxy units per M valence; and
M is selected from the alkali or alkaline-earth metal groups.

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

One aspect of the invention provides a system and method for disposing of per- and/or polyfluoroalkyl substances (PFAS) with reduced emissions of gaseous PFC, such as CF₄ and C₂F₆. In certain embodiments, the PFAS is a single per- and/or polyfluorinated compound or a mixture of several per- and/or polyfluorinated compounds. The current invention also pertains to a method of adding at least one solvent system to the PFAS and applying several heating temperatures in the degradation process. More specifically, the subject matter disclosed herein relates to a system and method that can be used for reducing emissions of gaseous perfluorinated compounds (PFCs) during thermal treatment of PFAS.

Various types of PFAS can be treated with the batch system according to the present invention, for example perfluorooctanoic acid (PFOA). Although the system and method are typically applied to PFAS, and PFAS will be discussed throughout the present disclosure, the system and method can be used to dispose of any type of fluorocarbon or fluorinated material. For example, the system and method disclosed herein can be applied to any source of water that contains fluorocarbon or fluorinated materials, in order to degrade and dispose of the fluorocarbon or fluorinated materials, for example per- and polyfluoroalkyl substances (PFAS) contained in contaminated water, such as “leachate water”. The system and method destroy the carbon-fluorine bonds and convert the organic fluorine present in the fluorocarbon or other fluorinated material to inorganic fluoride.

The method for disposing the PFAS first includes placing the water containing PFAS in a batch system, more specifically in a batch reactor. Next, a hydroxide base is added to the batch reactor, for example potassium hydroxide (KOH), calcium hydroxide (Ca(OH)₂), cesium hydroxide (CsOH), lithium hydroxide (LiOH), sodium hydroxide (NaOH), and/or strontium hydroxide (Sr(OH)₂). A solvent system including one or more solvents is also optionally added to the batch reactor and combined with the water containing PFAS and hydroxide base to form a solution. The solvent system can include diglyme, polyethers, polyether alcohol, a polyethylene glycol selected from ethylene glycol and PEG50 through PEG3350, N-methylpyrrolidine, cyrene and/or water (“solvent”)

An example solvent is a polyethylene glycol ether. Water may also be present optionally as a co-solvent in the batch reactor. The PFAS may be provided in an aqueous film-forming foam (AFFF), also known as a fire-fighting foam. The AFFF is typically a suspension composition. Also, some of the solvents listed above may already be present in the AFFF suspension composition. Preferably, the ratio of water to hydroxide base, such as KOH (water:KOH), in w/w % ranges from 1:1 to 1:0.5. Typically, between 0.010 Liters to 0.5 Liters of hydroxide base, and optionally 0.010 Liter to 0.5 Liter of solvent, is used for each Liter of water.

The PFAS is typically maintained in the batch reactor at a temperature of ranging from room temperature for several days, or 100° C. and 200° C. for at least 2 hours, for example 3 to 5 hours, or up to 8 hours to defluorinate the PFAS and produce a defluorinated waste product consisting of inorganic fluoride. Some types of PFAS, such as perfluorooctyl sulfonate (PFOS), may require higher temperatures and longer times in the reactor, for example temperatures up to but not limited to 300° C. According to other embodiments, the temperature of the batch system may be less than 100° C., for example room temperature or 50° C. up to 100° C. When the temperature of the batch system is lower, the time required to defluorinate the PFAS- and produce a defluorinated waste product consisting of an inorganic fluoride is longer. The defluorinated waste product produced may typically include polyethylene glycol and/or the solvent used in the reactor, formate, carbonate, oxalate and/or glycolate, and inorganic fluoride(s) wherein the composition of the inorganic fluoride, i.e. potassium fluoride, sodium fluoride, lithium fluoride and/or calcium fluoride or combinations thereof, etc., depends on the hydroxide base or mixture of hydroxide bases used in the batch system, The defluorinated waste product can be further incinerated without significant emissions of the harmful gaseous PFCs.

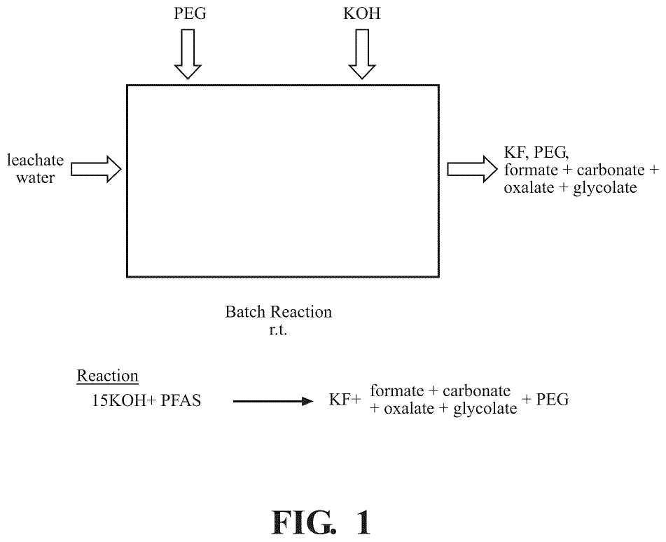

FIG. 1 illustrates the batch system and method according to an example embodiment.

According to this example, water containing PFOA is placed in the batch reactor along with PEG and potassium hydroxide (KOH). The PEG is preferably PEG200 which has a molar mass of 190-210 g/mol and a chemical formula of H—(O—CH₂CH₂)ₙ—OH, where n=8.2 to 9.1. It is believed that the PEG200 could be replaced optionally with diglyme, polyethers, polyether alcohol, a polyethylene glycol selected from ethylene glycol and PEG50 through PEG3350, N-methylpyrrolidine, cyrene and/or water. Alternatively, no solvent is added since some of these solvents may already be present in the AFFF suspension composition, and the KOH could be replaced with another hydroxide base comprised of but not limited to sodium, calcium, lithium, strontium or cesium or optionally mixtures thereof, etc.

According to this example, the PFOA is allowed to react in the batch system at a temperature of 180° C. to 200° C. for approximately 4 hours at ambient pressure. The resulting defluorinated waste product includes the product generated potassium fluoride (KF), PEG200, unreacted excess potassium hydroxide (KOH), and carbonate and/or formate and/or oxalate and/or glycolate or mixtures thereof. The chemical reaction taking place in the batch system of FIG. 1 includes:

15KOH+C₈F₁₅O₂H→15KF+carbonate+formate+oxalate+glycolate.

After the batch process, the defluorinated waste product can be thermally treated, for example by incineration, with reduced emissions of the hazardous gaseous PFCs, such as CF₄ and C₂F₆.

Before incineration, some of the components present in the defluorinated waste product can be recycled or removed and disposed of without thermal treatment. For example, according to one embodiment, the PEG200 is removed from the defluorinated waste product and recycled. The recycled PEG200 can be used in future batch systems.

Another aspect of the invention is the capability of reusing the unreacted components in the process of defluorination of the AFFF suspension or other PFAS containing substances. Upon reaction completion, the resulting mixture is treated with caustic lime (calcium hydroxide) which reacts with the inorganic fluoride species that are produced during the AFFF suspension components defluorination reaction. The resulting calcium fluoride from the caustic lime treatment precipitates out of the entire combination of components. Upon filtration of the precipitated calcium fluoride, the filtrate of the reaction mixture can then be reused in a subsequent process.

### EXPERIMENT

### General Procedure for the Defluorination Reaction.

Leachate water was treated with a hydroxide base (sodium hydroxide, potassium hydroxide, strontium hydroxide, calcium hydroxide or combinations thereof in various ratios) either neat or in the presence of a solvent, diglyme, polyethers, polyether alcohol, a polyethylene glycol selected from ethylene glycol and PEG50 through PEG3350, N-methylpyrrolidine, cyrene and/or water in various ratios at 150° C. to 200° C. for 4 hours. The resulting reaction mixture was allowed to cool to room temperature. The reaction material was analyzed by 19F NMR.

### Representative Example 1. Solvent Assisted

In a 40 mL vial with a screwcap, PEG200 (1.5 equivalents w/w, 6 g) was added to crushed potassium hydroxide pellets (1.5 equivalents w/w, 6 g) followed by the addition of Leachate water (1 equivalent w/w, 4 g). The vial was immersed in a pre-heated sand bath (hot plate T: 150° C. to 200° C.) and allowed to react for 4 hours. The resulting pale yellowish clear solution was allowed to cool to room temperature followed by ¹⁹F NMR analysis. The reaction mixture showed the presence of only inorganic potassium fluoride.

### Representative Example 2. Neat. No Additional Solvent

In a 40 mL vial with a screwcap, leachate water (1 equivalent w/w, 4 g) was added to crushed potassium hydroxide pellets (1.5 equivalents w/w, 6 g). The vial was immersed in a pre-heated sand bath (hot plate T: 150° C. to 200° C.) and allowed to react for 4 hours. The resulting pale yellowish clear solution was allowed to cool to room temperature, followed by ¹⁹F NMR analysis. The reaction mixture showed the presence of only inorganic potassium fluoride.

An experiment was conducted to confirm that the batch system and method disclosed herein can successfully destroy PFOA and can convert the organic fluorine present in the PFOA to innocuous inorganic fluoride.

The experiment included dissolving reagent grade perfluorooctanoic acid (PFOA-CAS 335-67-1) in polyethylene glycol (PEG200). Crushed potassium hydroxide pellets (KOH) were added to produce a solution containing approximately 50% PEG200 and 50% KOH by weight. This solution was stirred and heated to approximately 150° C. to 200° C. for four hours in a closed 40-mL screwed cap vial.

The reaction mixture was then analyzed by ¹⁹Fluorine-Nuclear Magnetic Resonance (NMR). Before the reaction was initiated by heating, the NMR spectra showed peaks characteristic of the PFOA component. All PFOA peaks had disappeared and only a large fluoride peak remained. The ¹⁹F-NMR results provided a qualitative indication that PFOA was destroyed, and organic fluorine was converted into innocuous inorganic fluoride. These qualitative NMR results indicated the effectiveness of the batch system and method disclosed herein should be at least 98%.

In summary, in accordance with the purposes of the disclosed materials and methods, as embodied and broadly described herein, the disclosed subject matter, in one aspect, relates to the composition and methods of defluorination of water contaminated with fluorocarbon or fluorinated material, such as leachate water including PFAS contaminants generating inorganic fluoride in the form of a salt. Moreover, it relates to methods of reducing emissions of gaseous perfluorinated compounds (PFCs) during thermal treatment of leachate water. In specific aspects, the disclosed subject matter relates to the selection of materials for a greener process.

Certain embodiments of this invention provide a composition comprising at least one solvent system and a strong base, wherein said solvent system is comprised of at least one of diglyme, polyethers, polyether alcohol, a polyethylene glycol selected from ethylene glycol and PEG50 through PEG3350, N-methylpyrrolidine, cyrene and/or water, and wherein the hydroxide base is comprised of potassium hydroxide, sodium hydroxide, cesium hydroxide, lithium hydroxide, strontium hydroxide, and/or calcium hydroxide either by themselves or in combination at different compositions in w/w % according to reaction scheme I.

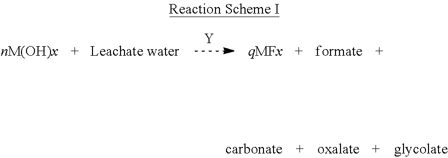

wherein;
n is the minimum amount of molar equivalents to degrade the organic fluorine in leachate water;
M is comprised of potassium, sodium, cesium, lithium, strontium or calcium;
q is the maximum amount of molar equivalents generated by degradation of the organic fluorine in leachate water;
x is the number of hydroxy (OH) and fluoride (F) units per M valence; and,
Y is comprised of the list of “solvent”.

In another embodiment, these compositions as described hereinabove, do not include addition of the solvent according to the reaction scheme II.

nM(OH)x+Leachate water--->qMFx+formate+carbonate+oxalate+glycolate Reaction Scheme II
wherein;
n is the minimum amount of molar equivalents to degrade the organic fluorine in leachate water; M is comprised of potassium, sodium, cesium, lithium, strontium or calcium;
q is the maximum amount of molar equivalents generated by degradation of the organic fluorine in leachate water;
x is the number of hydroxy (OH) and fluoride (F) units per M valence; and,

A system and method for disposing per- and polyfluoroalkyl substances (PFAS) contained in leachate water with reduced emissions of gaseous PFCs is provided. The leachate water is placed in a batch system containing a hydroxide base with or without the presence of the solvent system, such as diglyme, polyethers, polyether alcohol, a polyethylene glycol selected from ethylene glycol and PEG50 through PEG3350, N-methylpyrrolidine, cyrene and/or water to defluorinate the fluorocarbon(s) present as PFAS in the leachate water forming a defluorinated waste product such as formate, carbonate, oxalate, and glycolate. After defluorinating, the fluorocarbon compounds mixture of the PFAS in leachate water, a thermal treatment, for example incineration, may be performed on the defluorinated waste product with reduced emissions of the harmful gaseous PFCs.

Obviously, many modifications and variations of the present invention are possible in light of the above teachings and may be practiced otherwise than as specifically described while within the scope of the following disclosure and claims.

### LITERATURE CITED

1. Masoner, J. Environ. Sci.: Water Res. Technol., 2020, 6, 1300-1311.
2. U.S. Environmental Protection Agency, Advancing Sustainable Materials Management: 2015 Fact Sheet, 2015_smm_msw_factsheet_07242018_fnl_508_002.pdf (accessed Jun. 26, 2019).
3. H. Hamid, L. Y. Li and J. R. Grace, Review of the Fate and Transformation of Per-and Polyfluoroalkyl Substances (PFASs) in Landfills, Environ. Pollut., 2018, 235, 74-84.
4. B. O. Clarke, T. Anumol, M. Barlaz and S. A. Snyder, Investigating Landfill Leachate as a Source of Trace Organic Pollutants, Chemosphere, 2015, 127, 269-275.
5. J. R. Masoner, D. W. Kolpin, E. T. Furlong, I. M. Cozzarelli and J. L. Gray, Landfill Leachate as a Mirror of Today's Disposable Society: Pharmaceuticals and Other Contaminants of Emerging Concern in Final Leachate from Landfills in the Conterminous United States, Environ. Toxicol. Chem., 2016, 3, 906-918.
6. J. R. Masoner, D. W. Kolpin, E. T. Furlong, I. M. Cozzarelli, J. L. Gray and E. A. Schwab, Contaminants of Emerging Concern in Fresh Leachate from Landfills in the Conterminous United States, Environ. Sci.: Processes Impacts, 2014, 16, 2335-2354.
7. J. R. Lang, B. M. Allred, J. A. Field, J. W. Levis and M. A. Barlaz, National Estimate of Per-and Polyfluoroalkyl Substance (PFAS) Release to U.S. Municipal Landfill Leachate, Environ. Sci. Technol., 2017, 51, 2197-2205.
8. T. Eggen, M. Moeder and A. Arukwe, Municipal Landfill Leachates: A Significant Source for New and Emerging Pollutants, Sci. Total Environ., 2010, 408, 5147-5157.
9. W. J. Andrews, J. R. Masoner and I. M. Cozzarelli, Emerging Contaminants at a Closed and an Operating Landfill in Oklahoma, Groundwater Monit. Rem., 2012, 32, 120-130.
10. Z. Wei, T. Xu and D. Zhao, Treatment of Per-and Polyfluoroalkyl Substances in Landfill Leachate: Status, Chemistry and Prospects, Environ. Sci.: Water Res. Technol., 2019, 5,1814-1835.
11. A. Vellinga, S. Cormican, J. Driscoll, M. Furey, M. O'Sullivan and M. Cormican, Public Practice Regarding Disposal of Unused Medicines in Ireland, Sci. Total Environ., 2014, 478, 98-102.
12. G. Hawkins, The Performativity of Food Packaging: Market Devices, Waste Crisis and Recycling, Sociol. Rev., 2012, 60, 66-83.
13. M. Kotthoff, J. Müller, H. Jürling, M. Schlummer and D. Fiedler, Perfluoroalkyl and Polyfluoroalkyl Substances in Consumer Products, Environ. Sci. Pollut. Res., 2015, 22, 14546-14559.
14. B. D. Key, R. D. Howell and C. S. Criddle, Fluorinated Organics in the Biosphere, Environ. Sci. Technol., 1997, 31, 2445-2454.
15. K. Li, P. Gao, P. Xiang, X. Zhang, X. Cui and L. Q. Ma, Molecular Mechanisms of PFOA-Induced Toxicity in Animals and Humans: Implications for Health Risks, Environ. Int., 2017, 99, 43-54.
16. R. Crebelli, S. Caiola, L. Conti, E. Cordelli, G. De Luca, E. Dellatte, P. Eleuteri, N. Iacovella, P. Leopardi and F. Marcon, Can Sustained Exposure to PFAS Trigger a Genotoxic Response?A Comprehensive Genotoxicity Assessment in Mice after Subacute Oral Administration of PFOA and PFBA, Regul. Toxicol. Pharmacol., 2019, 106, 169-177.
17. Agency for Toxic Substances and Disease Registry, PFAS and Their Health Effects https://www.atsdr.cdc.gov/pfas/PFAS-health-effects.html (accessed Oct. 10, 2019).
18. K. Winkens, R. Vestergren, U. Berger and I. T. Cousins, Early Life Exposure to Per-and Polyfluoroalkyl Substances (PFASs): A Critical Review, Emerg. Contam. 2017, 3, 55-68.
19. M. I. Vasquez, A. Lambrianides, M. Schneider, K. Kummerer and D. Fatta-Kassinos, Environmental Side Effects of Pharmaceutical Cocktails: What We Know and What We Should Know, J. Hazard. Mater., 2014, 279, 169-189.
20. V. Futran Fuhrman, A. Tal and S. Amon, Why Endocrine Disrupting Chemicals (EDCs) Challenge Traditional Risk Assessment and How to Respond, J. Hazard. Mater., 2015, 286, 589-611.
21. B. G. Loganathan, K. S. Sajwan, E. Sinclair, K. Senthil Kumar and K. Kannan, Perfluoroalkyl Sulfonates and Perfluorocarboxylates in Two Wastewater Treatment Facilities in Kentucky and Georgia, Water Res., 2007, 41, 4611-4620.
22. M. M. Schultz, C. P. Higgins, C. A. Huset, R. G. Luthy, D. F. Barofsky and J. A. Field, Fluorochemical Mass Flows in a Municipal Wastewater Treatment Facility, Environ. Sci. Technol., 2006, 40, 7350-7357.
23. X. Dauchy, V. Boiteux, C. Bach, A. Colin, J. Hemard, C. Rosin and J.-F. Munoz, Mass Flows and Fate of Per-and Polyfluoroalkyl Substances (PFASs) in the Wastewater Treatment Plant of a Fluorochemical Manufacturing Facility, Sci. Total Environ., 2017, 576, 549-558.
24. R. Guo, W.-J. Sim, E.-S. Lee, J.-H. Lee and J.-E. Oh, Evaluation of the Fate of Perfluoroalkyl Compounds in Wastewater Treatment Plants, Water Res., 2010, 44, 3476-3486.
25. T.-T. Pham and S. Proulx, PCBs and PAHs in the Montreal Urban Community (Quebec, Canada) Wastewater Treatment Plant and in the Effluent Plume in the St Lawrence River, Water Res., 1997, 31, 1887-1896.
26. S. Sun, L. Jia, B. Li, A. Yuan, L. Kong, H. Qi, W. Ma, A. Zhang and Y. Wu, The Occurrence and Fate of PAHs over Multiple Years in a Wastewater Treatment Plant of Harbin, Northeast China, Sci. Total Environ., 2018, 624, 491-498.
27. A. M. Sadaria, S. D. Supowit and R. U. Halden, Mass Balance Assessmentfor Six Neonicotinoid Insecticides During Conventional Wastewater and Wetland Treatment: Nationwide Reconnaissance in United States Wastewater, Environ. Sci. Technol., 2016, 50, 6199-6206.
28. J. Sánchez-Avila, J. Bonet, G. Velasco and S. Lacorte, Determination and Occurrence of Phthalates, Alkylphenols, Bisphenol A, PBDEs, PCBs and PAHs in an Industrial Sewage Grid Discharging to a Municipal Wastewater Treatment Plant, Sci. Total Environ., 2009, 407, 4157-4167.
29. P. Gago-Ferrero, M. Gros, L. Ahrens and K. Wiberg, Impact of On-Site, Small and Large Scale Wastewater Treatment Facilities on Levels and Fate of Pharmaceuticals, Personal Care Products, Artificial Sweeteners, Pesticides, and Perfluoroalkyl Substances in Recipient Waters, Sci. Total Environ., 2017, 601-602, 1289-1297.
30. W. Zhang, Y. Zhang, S. Taniyasu, L. W. Y. Yeung, P. K. S. Lam, J. Wang, X. Li, N. Yamashita and J. Dai, Distribution and Fate of Perfluoroalkyl Substances in Municipal Wastewater Treatment Plants in Economically Developed Areas of China, Environ. Pollut., 2013, 176, 10-17.
31. Y. Yang, Y. S. Ok, K.-H. Kim, E. E. Kwon and Y. F. Tsang, Occurrences and Removal of Pharmaceuticals and Personal Care Products (PPCPs) in Drinking Water and Water/Sewage Treatment Plants: A Review, Sci. Total Environ., 2017, 596-597, 303-320.
32. Florida Department of Environmental Protection, General Facts and Statistics about Wastewater in Florida https://floridadep.gov/water/domestic-wastewater/content/general-facts-and-statistics-about-wastewater-florida.
33. Zhang, M. Waste Management Volume 155, 1 Jan. 2023, Pages 162-178.
34. Meegola, J. N. et al. Int. J. Environ. Res. Public Health 2022, 19(24), 16397.
35. Ebnesajjad, S. Introduction to Fluoropolymers; Elsevier: Amsterdam, The Netherlands, 2013, 17-35.
36. Camdzic, D. et al. J. Hazard. Mater. Lett. 2021, 2, 100023. https://www.sciencedirect.com/science/article/pii/S2666911021000113#bib0285
37. Arana Juve, J.-M. et al. Curr. Opin. Chem. Engin. 2023, 41, 100943.
38. Buck, R. C. et al. Integr. Environ. Assess. Manag., 2011, 7(4), 513-541.
39. Ying, L. Occup. Environ. Med., 2018, 75(1), 46-51.
40. Kelly, B. C. et al. Environ. Sci. Technol., 2009, 43(11), 4037-4043.
41. Jiang, Q. et al. Toxicological Effects of Perfluoroalkyl and Polyfluoroalkyl Substances 2015, 177-201.
42. Abbott, B. D. et al. Reprod. Toxicol., 2012, 33(4), 491-505.
43. Leeson, A. et al., Environ. Toxicol. Chem. 2021. 40, 24-36.
44. Wang, Z. et al., Environ. Int. 2013, 60, 242-8; Vierke, L. et al., Environ. Sci. Eur. 2012, 24, 16; Leung, S. C. E. et al., Sci. Total Environ. 2022, 827, 153669. https://comptox.epa.gov/dashboard/chemical-lists/PFASSTRUCT; https://comptox.epa.gov/dashboard/chemical-lists/PFASDEV
45. Krafft, M. P. et al. Curr. Opin. Colloid Interface Sci. 2015, 20, 192-212; Ojo, A. F. et al. J. Hazard. Mater. 2021, 407, 12486.
46. https://www.whitehouse.gov/wp-content/uploads/2023/03/OSTP-March-2023-PFAS-Report.pdf
47. Int. J. Environ. Res. Public Health 2022, 19(24), 16397.
48. EPA. Per- and Polyfluoroalkyl Substances (PFAS): Incineration to Manage PFAS Waste Streams Background. US EPA Tech. Brief 2020.
49. Water Res. 2007, 41, 4611-4620.
50. Waste Manag. 2020, 107, 191-200.
51. Sci. Total Environ. 2020, 705, 135832.
52. Water Environ. Res. 2021, 93, 826-843.
53. https://cen.acs.org/environment/persistent-pollutants/Incincerators-spread-break-down-PFAS/98/web/2020/04
54. Stoiber, T.; Evans, S.; Naidenko, O. V. Disposal of products and materials containing per-and polyfluoroalkyl substances (PFAS): A cyclical problem. Chemosphere 2020, 260, 127659.
55. Crunden, E. A. Defense Department Hits the Brakes on PFAS Incineration. Available online: https://www.eenews.net/articles/defense-department-hits-the-brakes-on-pfas-incineration/
56. U.S. C. H. R.2591 PFAS Waste Incineration Ban Act of 2019. 2019. Available online: https://www.govinfo.gov/
57. U.S. C. Public Law 116-92-National Defense Authorization Actfor Fiscal Year2020. 2019. Available online: https://www.govinfo.gov/
58. Horst, J.; McDonough, J.; Ross, I.; Houtz, E. Understanding and managing the potential by-products of PFAS destruction. Groundw. Monit. Remediat. 2020, 40, 17-27.
59. Stockenhuber, S.; Weber, N.; Dixon, L.; Lucas, J.; Grimison, C.; Bennett, M.; Stockenhuber, M.; Mackie, J.; Kennedy, E. Thermal Degradation of Perfluorooctanoic Acid (PFOA). In Proceedings of the 16th International Conference on Environmental Science and Technology, Rhodes, Greece, 4-7 Sep. 2019.
60. Altarawneh, M.; Almatarneh, M. H.; Dlugogorski, B. Z. Thermal decomposition of perfluorinated carboxylic acids: Kinetic model and theoretical requirements for PFAS incineration. Chemosphere 2022, 286, 131685.
61. Sasi, P. C.; Alinezhad, A.; Yao, B.; Kubitovi, A.; Golovko, S. A.; Golovko, M. Y.; Xiao, F. Effect of granular activated carbon and other porous materials on thermal decomposition of per-and polyfluoroalkyl substances: Mechanisms and implications for water purification. Water Res. 2021, 200, 117271.
62. Xiao, F.; Sasi, P. C.; Yao, B.; Kubátová, A.; Golovko, S. A.; Golovko, M. Y.; Soli, D. Thermal decomposition of PFAS: Response to comment on “thermal stability and decomposition of perfluoroalkyl substances on spent granular activated carbon”. Environ. Sci. Technol. Lett. 2021, 8, 364-365.
63. Crownover, E.; Oberle, D.; Kluger, M.; Heron, G. Perfluoroalkyl and polyfluoroalkyl substances thermal desorption evaluation. Remediat. J. 2019, 29, 77-81.
64. Yamada, T.; Taylor, P. H.; Buck, R. C.; Kaiser, M. A.; Giraud, R. J. Thermal degradation of fluorotelomer treated articles and related materials. Chemosphere 2005, 61, 974-984.
65. Taylor, P. H.; Yamada, T.; Striebich, R. C.; Graham, J. L.; Giraud, R. J. Investigation of waste incineration of fluorotelomer-based polymers as a potential source of PFOA in the environment. Chemosphere 2014, 110, 17-22.
66. Vidonish, J. E.; Zygourakis, K.; Masiello, C. A.; Sabadell, G.; Alvarez, P. J. J. Thermal treatment of hydrocarbon-impacted soils: A review of technology innovation for sustainable remediation. Engineering 2016, 2, 426-437.
67. Wang, J.; Lin, Z.; He, X.; Song, M.; Westerhoff, P.; Doudrick, K.; Hanigan, D. Critical review of thermal decomposition of per-and polyfluoroalkyl substances: Mechanisms and implications for thermal treatment processes. Environ. Sci. Technol. 2022, 56, 5355-5370.
68. Aleksandrov, K.; Gehrmann, H. J.; Hauser, M.; Matzing, H.; Pigeon, D.; Stapf, D.; Wexler, M. Waste incineration of polytetrafluoroethylene (PTFE) to evaluate potential formation of per-and poly-fluorinated alkyl substances (PFAS) in Flue Gas. Chemosphere 2019, 226, 898-906.
69. Garciá, A. N.; Viciano, N.; Font, R. Products obtained in the fuel-rich combustion of PTFE at high temperature. J. Anal. Appl. Pyrolysis 2007, 80, 85-91.
70. Wang, F.; Shih, K.; Lu, X.; Liu, C. Mineralization behavior of fluorine in perfluorooctanesulfonate (PFOS) during thermal treatment of lime-conditioned sludge. Environ. Sci. Technol. 2013, 47, 2621-2627.

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

What is claimed is:

1. A method for disposing a perfluoroalkyl and/or polyfluoroalkyl substance (PFAS) contained in water, comprising the steps of:
placing the water, a hydroxide base, and optionally a solvent system in a batch reactor to form a suspension, and heating the suspension in the batch reactor to produce a defluorinated waste product.

2. The method of claim 1 including placing the water in the batch reactor, wherein the water contains the PFAS and optionally the solvent system.

3. The method of claim 1, wherein the water containing the PFAS, hydroxide base, and optionally the solvent system are placed in the batch reactor and allowed to react with one another for a time of about 0.5 hours to about 240 hours; and the heating step includes heating the suspension to a temperature ranging from about 25° C. to about 400° C.

4. The method of claim 1, wherein the hydroxide base includes at least one of potassium hydroxide (KOH), calcium hydroxide (Ca(OH)₂), cesium hydroxide (CsOH), lithium hydroxide (LiOH), sodium hydroxide (NaOH), strontium hydroxide (Sr(OH)₂), and mixtures thereof.

5. The method of claim 1, wherein the solvent system is added and includes at least one of diglyme, polyethers, polyether alcohol, a polyethylene glycol selected from ethylene glycol and PEG50 through PEG3350, N-methylpyrrolidine, cyrene and/or water.

6. The method of claim 1, wherein the hydroxide base includes a mixture of two hydroxide bases, and the hydroxide bases are in a ratio of about 1:99 w/w % to about 99:1 w/w %.

7. The method of claim 6, wherein the hydroxide bases are in a ratio of about 25:75 w/w % to about 75:25 w/w %.

8. The method of claim 6, wherein the hydroxide bases are in a ratio of about 50:50 w/w %.

9. The method of claim 1, wherein the solvent system and the water are provided by a single substance, and no additional solvent is added.

10. The method of claim 1, wherein the water, hydroxide base, and solvent system are placed in the batch reactor and allowed to react with one another for a time of about 0.5 hours to about 240 hours.

11. The method of claim 10, wherein the water, hydroxide base, and solvent system are placed in the batch reactor and allowed to react with one another for a time of about 3 hours to about 120 hours.

12. The method of claim 1, wherein the water, hydroxide base, and solvent system are placed in the batch reactor and allowed to react with one another for a time of about 4 hours to about 60 hours.

13. The method of claim 1, wherein the water, hydroxide base, and solvent system are placed in the batch reactor and allowed to react with one another for a time of about 4 hours to about 24 hours.

14. The method of claim 1, wherein the water, hydroxide base, and solvent system are placed in the batch reactor and allowed to react with one another for a time of about 4 hours to about 10 hours.

15. The method of claim 1, wherein the water, hydroxide base, and solvent system are placed in the batch reactor and allowed to react with one another for a time of about 4 hours.

16. The method of claim 1, wherein the water, hydroxide base, and solvent system are placed in the batch reactor and allowed to react with one another for a time of about 8 hours.

17. The method of claim 1, wherein the heating step includes heating the suspension to a temperature of about 25° C. to about 300° C.

18. The method of claim 1, wherein the heating step includes heating the suspension to a temperature of about 100° C. to about 300° C.

19. The method of claim 1, wherein the heating step includes heating the suspension to a temperature of about 100° C. to about 200° C.

20. The method of claim 1, wherein the heating step includes heating the suspension to a temperature of about 150° C. to about 200° C.

21. The method of claim 1, wherein the heating step includes heating the suspension to a temperature of about 180° C.

22. The method of claim 1, wherein the hydroxide base is potassium hydroxide, the heating step includes heating to a temperature of about 180° C., the water, hydroxide base, and solvent system are placed in the batch reactor and allowed to react with one another for a time of about 4 hours, and the water and solvent system are provided by a single substance, and no additional solvent is added to the batch reactors.

23. The method of claim 1, wherein the hydroxide base is potassium hydroxide, the heating step includes heating to a temperature of about 180° C., the water, hydroxide base, and solvent system are placed in the batch reactor and allowed to react with one another for a time of about 8 hours, and the water and solvent system are provided by a single substance, and no additional solvent is added to the batch reactors.

24. The method of claim 1 including incinerating the defluorinated waste product.

25. The method of claim 1, wherein the water is leachate.

26. The method of claim 25, wherein a source of the leachate is a landfill.

27. A system comprising a batch reactor for disposing the water according to the method of claim 1.
